(19) United States
(12) Patent Application Publication
(10) Pub. No.: US 2014/0322271 A1
(43) Pub. Date: Oct. 30, 2014

(54) VACCINE COMPRISING A TLR-5 AGONIST AS ADJUVANT FOR USE IN CUTANEOUS IMMUNISATION

(71) Applicant: GlaxoSmithKline Biologicals S.A., Rixensart (BE)

(72) Inventors: Nathalie Marie-Josephe Garcon-Johnson, Rixensart (BE); Marcelle Paulette Van Mechelen, Rixensart (BE); Martin Plante, Quebec (CA)

(21) Appl. No.: 14/359,401

(22) PCT Filed: Nov. 19, 2012
(86) PCT No.: PCT/EP2012/072947

§ 371 Date: May 20, 2014

(30) Foreign Application Priority Data
Nov. 20, 2011 (GB) 1119999.9

(51) Int. Cl. A61K 39/29; A61K 39/39; A61K 9/00

(52) U.S. Cl. CPC A61K 39/292; A61K 9/0021; A61K 39/39; A61K 2039/54
USPC 424/227.1

(57) ABSTRACT

The present invention provides immunogenic compositions comprising one or more antigens and an adjuvant for use in cutaneous immunisation wherein said adjuvant is a TLR-5 agonist.

### FIELD OF THE INVENTION

The present invention provides immunogenic compositions for use in cutaneous immunisation comprising antigens and adjuvant, which adjuvant is a TLR-5 agonist.

### BACKGROUND TO THE INVENTION

There is in general a need to increase patient compliance with vaccination as well as to improve ease of manufacture and transport of vaccines whether prime or booster vaccination. Cutaneous immunisation can address some of these needs and can be used to administer antigens in combination with adjuvants to induce antigen-specific immune responses.

### SUMMARY OF THE INVENTION

It is an object of the invention to stimulate the immune response to a vaccine in a subject. The vaccine comprises an immunogenic composition comprising both antigen and adjuvant, and is administered cutaneously.

The adjuvant within the immunogenic composition is a TLR-5 agonist.

### BRIEF DESCRIPTION OF THE DRAWINGS

FIG. 1: Mouse immunogenicity data Results IgG anti-HBs 14 days post 2nd

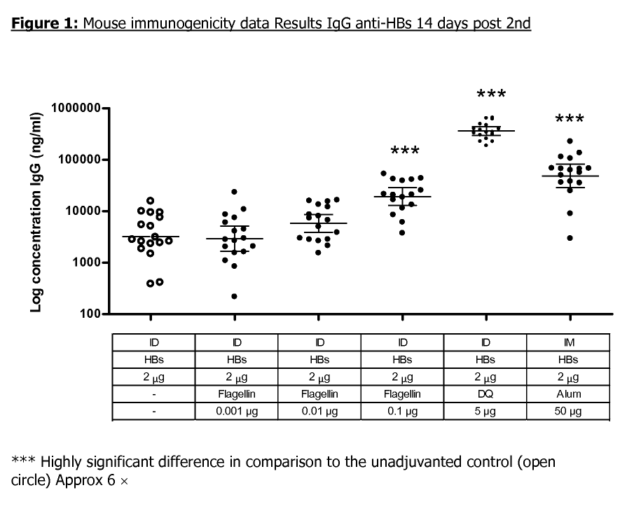

### DETAILED DESCRIPTION

In one embodiment, the present invention provides an? immunogenic composition comprising one or more antigens and an adjuvant for use in cutaneous immunisation wherein said adjuvant is a TLR-5 agonist.

In another embodiment is provided the use of an immunogenic composition comprising one or more antigens and an adjuvant in the manufacture of a medicament for cutaneous immunisation wherein said adjuvant is a TLR-5 agonist.

In another embodiment is provided a method of cutaneous immunisation comprising the steps of applying cutaneously to a subject an immunogenic composition comprising one or more antigens and an adjuvant wherein said adjuvant is a TLR-5 agonist.

The term cutaneously as used herein is intended to refer to the application of antigens into the dermis and/or epidermis of human skin. The present invention in particular, utilises a delivery system for cutaneous immunisation which induces an immune response in an animal or human although conventional methods of administration are also encompassed.

Cutaneous application of an immunogenic composition comprising at least one antigen and an adjuvant, wherein the adjuvant is a TLR-5 agonist may be performed by using any cutaneous method known to the skilled person which include but is not limited to delivery using a short needle device (a device comprising a needle that is between about 1 and about 2 mm in length) or delivery using a skin patch.

Suitable devices for use with the cutaneous vaccines described herein include short needle devices such as those described in U.S. Pat. No. 4,886,499, U.S. Pat. No. 5,190,521, U.S. Pat. No. 5,328,483, U.S. Pat. No. 5,527,288, U.S. Pat. No. 4,270,537, U.S. Pat. No. 5,015,235, U.S. Pat. No. 5,141,496, U.S. Pat. No. 5,417,662 and EP1092444. Cutaneous vaccines may also be administered by devices which limit the effective penetration length of a needle into the skin, such as those described in WO99/34850, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Jet injection devices are described for example in U.S. Pat. No. 5,480,381, U.S. Pat. No. 5,599,302, U.S. Pat. No. 5,334,144, U.S. Pat. No. 5,993,412, U.S. Pat. No. 5,649,912, U.S. Pat. No. 5,569,189, U.S. Pat. No. 5,704,911, U.S. Pat. No. 5,383,851, U.S. Pat. No. 5,893,397, U.S. Pat. No. 5,466,220, U.S. Pat. No. 5,339,163, U.S. Pat. No. 5,312,335, U.S. Pat. No. 5,503,627, U.S. Pat. No. 5,064,413, U.S. Pat. No. 5,520, 639, U.S. Pat. No. 4,596,556 U.S. Pat. No. 4,790,824, U.S. Pat. No. 4,941,880, U.S. Pat. No. 4,940,460, WO 97/37705 and WO 97/13537. Also suitable are ballistic powder/particle delivery devices which use compressed gas to accelerate vaccine in powder form through the outer layers of the skin to the dermis. Additionally, conventional syringes may be used in the classical mantoux method of cutaneous administration. However, the use of conventional syringes requires highly skilled operators and thus devices which are capable of accurate delivery without a highly skilled user are preferred. Accordingly, in one embodiment, there is provided immunogenic compositions of the invention for use in cutaneous immunisation wherein the immunogenic composition is not administered by the mantoux method using a conventional syringe.

In a particular embodiment of the invention, there is provided a patch comprising immunogenic compositions of the invention as described herein. The patch will generally comprise a backing plate which includes a solid substrate (e.g. occlusive or nonocclusive surgical dressing). Patches of the invention deliver the antigen and adjuvant of the invention to the dermis or epidermis. Accordingly, patches of the invention comprise one or more microprojections adapted to deliver immunogenic composition of the invention to the epidermis or dermis. In one embodiment of the invention the one or more microprojections are between 10 μm and 2 mm, for example 20 μm to 500 μm, 30 μm to 1 mm, 100 to 200, 200 to 300, 300 to 400, 400 to 500, 500 to 600, 600 to 700, 700, 800, 800 to 900, 100 μm to 400 μm, in particular between about 200 μm and 300 μm or between about 150 μm and 250 μm.

In particular embodiment, the patches of the present invention comprise a plurality of microprojections. In a particular embodiment, patches of the invention comprise between 2 and 5000 microneedles for example between 1000 and 2000, microprojections.

In a particular embodiment, the microprojections are separated by a distance of between about 50 μm and 1000 μm.

The microprojections may be of any shape suitable for piercing the stratum corneum, epidermis and/or dermis and delivery and antigen and adjuvant to the epidermis or dermis. Microprojections may be shaped as disclosed in WO2000/074765 and WO2000/074766 for example. The microprojections may have an aspect ratio of at least 3:1 (height to diameter at base), at least about 2:1, or at least about 1:1. A particularly preferred shape for the microprojections is a cone with a polygonal bottom, for example hexagonal or rhombus-shaped. Other possible microprojection shapes are shown, for example, in U.S. Published Patent App. 2004/0087992. In a particular embodiment, microprojections of the invention have a shape which becomes thicker towards the base.

The number of microprotrusions in the array is preferably at least about 100, at least about 500, at least about 1000, at least about 1400, at least about 1600, or at least about 2000. The area density of microprotrusions, given their small size, may not be particularly high, but for example the number of microprotrusions per cm2 may be at least about 50, at least about 250, at least about 500, at least about 750, at least about 1000, or at least about 1500.

In one embodiment of the invention the antigen and adjuvant of the invention are delivered to the host within 5 hours of placing the patch on the skin of the host, for example, within 4 hours, 3 hours, 2 hours, 1 hour or 30 minutes. In a particular embodiment of the invention, the antigen and adjuvant of the invention delivered within 20 minutes of placing the patch of the skin, for example within 30 seconds 1, 2, 3, 4, 5, 6, 7, 8, 9, 10, 11, 12, 13, 14, 15, 16, 17, 18 or 19 minutes.

The microprojections can be made of any suitable material known to the skilled person. In a particular embodiment at least part of the microprojections are biodegradable, in particular the tip of the microprojection outer most layer of the microprojection. In a particular embodiment substantially all the microprojection is biodegradable. The term “biodegradable” as used herein means degradable under expected conditions of in vivo use (e.g. insertion into skin), irrespective of the mechanism of biodegradation. Exemplary mechanisms of biodegradation include disintegration, dispersion, dissolution, erosion, hydrolysis, and enzymatic degradation. By substantially all, it is meant that at least 70% of the microprojection is biodegradable, for example, at least 75%, 80%, 85%, 90 or at least 95% biodegradable

In a particular embodiment, biodegradable microprojections comprise a biodegradable polymer. For example, suitable biocompatible, biodegradable, or bioerodible polymers include poly(lactic acid) (PLA), poly(glycolic acid) (PGA), poly(lactic acid-co-glycolic acid)s (PLGAs), polyanhydrides, polyorthoesters, polyetheresters, polycaprolactones (PCL), polyesteramides, poly(butyric acid), poly(valeric acid), polyvinylpyrrolidone (PVP), polyvinyl alcohol (PVA), polyethylene glycol (PEG), block copolymers of PEG-PLA, PEG-PLA-PEG, PLA-PEG-PLA, PEG-PLGA, PEG-PLGA-PEG, PLGA-PEG-PLGA, PEG-PCL, PEG-PCL-PEG, PCL-PEG-PCL, copolymers of ethylene glycol-propylene glycol-ethylene glycol (PEG-PPG-PEG, trade name of Pluronic® or Poloxamer®), dextran, hetastarch, tetrastarch, pentastarch, hydroxyethyl starches, cellulose, hydroxypropyl cellulose (HPC), sodium carboxymethyl cellulose (Na CMC), thermosensitive HPMC (hydroxypropyl methyl cellulose), polyphosphazene, hydroxyethyl cellulose (HEC), other polysaccharides, polyalcohols, gelatin, alginate, chitosan, hyaluronic acid and its derivatives, collagen and its derivatives, polyurethanes, and copolymers and blends of these polymers. A preferred hydroxyethyl starch may have a degree of substitution of in the range of 0-0.9.

In a particular embodiment the biodegradable portion of the microprojections comprise the antigen and/or adjuvant. The antigen and/or adjuvant may be found in separate microprojections for example about 90%, 80%, 70%, 60%, 50%, 40%, 30% of microprojections may comprise antigen and 10%, 20%, 30% , 40%, 50%, 60% or 70% of microprojections may comprise adjuvant, respectively. In a particular embodiment there is provided a patch comprising one or more, in particular a plurality, biodegradeable microprojections that comprise immunogenic compositions as described herein. Examples of microprojections comprising actives such as antigens are disclosed in WO2008/130587 and WO2009/048607. Methods of manufacture of metabolisable microneedles are disclosed in WO2008/130587 and WO2010/124255.

In a further embodiment, the adjuvant and antigen are coated on one or more microprojections. Coating can be performed any method known to the skilled person for example by the methods disclosed in WO06/055844, WO06/055799.

The antigen and/or adjuvant may be coated on separate microprojections 90%, 80%, 70%, 60%, 50%, 40%, 30% of microprojections may be coated with antigen and 10%, 20%, 30% , 40%, 50%, 60% or 70% of microprojections may be coated with adjuvant, respectively.

The patches of the invention may be applied to the skin of the wearer by any means for example by placing the patches on the skin with a hand. In a particular embodiment, the patch of the invention is applied to the skin using an applicator, for example applicators described in WO2008/091602. In particular the application comprises a means for ensuring that the patch has been applied to the skin with sufficient pressure to ensure that the one or more microprojections penetrate the stratum corneum, epidermis and/or dermis, for example a device that makes an audible sound when sufficient pressure has been applied.

Patches of the invention may also comprise an adhesive to aid retention of the patch on the skin during release/delivery of the antigen and adjuvant into the dermis and/or epidermis.

The immunogenic compositions of the present invention comprise both an antigen and an adjuvant. An adjuvant is a component of the immunogenic composition which assists in inducing an immune response to the antigen. In the present invention, the immunogenic composition for use in cutaneous immunisation comprises an adjuvant which is a TLR-5 agonist.

Said TLR-5 agonist may be flagellin or may be a fragment of flagellin which retains TLR-5 agonist activity. The flagellin can include a polypeptide selected from the group consisting of H. pylori, S. typhimurium, V. cholera, S. marcesens, S. flexneri, T. pallidum, L. pneumophilia, B. burgdorferei; C. difficile, R. meliloti, A. tumefaciens; R. lupine; B. clarridgeiae, P. mirabilis, B. subtilus, L. moncytogenes, P. aeruginoa and E. coli.

In a particular embodiment, the flagellin is selected from the group consisting of S. typhimurium flagellin B (Genbank Accession number AF045151), a fragment of S. typhimurium flagellin B, E. coli FliC. (Genbank Accession number AB028476); fragment of E. coli FliC; S. typhimurium flagellin FliC (ATCC14028) and a fragment of S. typhimurium flagellin FliC.

In a particular embodiment, said TLR-5 agonist is a truncated flagellin as described in WO2009/156405 i.e. one in which the hypervariable domain has been deleted. In one aspect of this embodiment, said TLR-5 agonist is selected from the group consisting of: FliC_{Δ174-400}; FliC_{Δ161-405} and FliC_{Δ138-405}.

In a further embodiment, said TLR-5 agonist is a flagellin as described in WO2009/128950.

If the TLR-5 agonist is a fragment of a flagellin, it will be understood that said fragment will retain TLR5 agonist activity, and must therefore retain the portion of its sequence responsible for TLR-5 activation. It is known by the person skilled in the art that the NH₂ and COOH terminal domains of flagellin are important for TLR-5 interaction and activation, in particular for example aa 86-92 in Salmonella.

In one embodiment, the immunogenic composition comprises a TLR-5 agonist as defined herein and no other adjuvant. In another embodiment, the immunogenic composition comprises a TLR-5 agonist and one or more other adjuvants. In one aspect of this embodiment, said one or more other adjuvants are selected from the group consisting of TLR-4 agonists, TLR 7/8 agonists and immunologically active saponin fractions.

A particularly suitable saponin for use in the present invention is Quil A and its derivatives. Quil A is a saponin preparation isolated from the South American tree Quillaja Saponaria Molina and was first described by Dalsgaard et al. in 1974 (“Saponin adjuvants”, Archiv. für die gesamte Virusforschung, Vol. 44, Springer Verlag, Berlin, p 243-254) to have adjuvant activity. Purified fragments of Quil A have been isolated by HPLC which retain adjuvant activity without the toxicity associated with Quil A (EP 0 362 278), for example QS7 and QS21 (also known as QA7 and QA21). QS-21 is a natural saponin derived from the bark of Quillaja saponaria Molina, which induces CD8+ cytotoxic T cells (CTLs), Th1 cells and a predominant IgG2a antibody response and is a preferred saponin in the context of the present invention. In a particular embodiment of the invention the immunologically active saponin is QS21. In particular the QS21 in substantially pure form, that is to say, the QS21 is at least 90% pure, preferably at least 95% pure and most preferably at least 98% pure. In a particular embodiment of the invention QS21 is formulated with a sterol. Preferred sterols include β-sitosterol, stigmasterol, ergosterol, ergocalciferol and cholesterol. These sterols are well known in the art, for example cholesterol is disclosed in the Merck Index, 11th Edn., page 341, as a naturally occuring sterol found in animal fat. In a particular embodiment of the invention the sterol is cholesterol. In a particular embodiment of the invention, the ratio of QS21 to cholesterol is between 1:100 and 1:1, in particular between 1:2 and 1:10, for example 1:5.

TLR-4 agonists are agonists of Toll Like receptor 4, a member of the Toll Like Receptor family. This is a well known family of receptors, all of which are involved in some way in immune responses. In one embodiment, the TLR-4 agonist is a lipopolysaccharide, suitably a non-toxic derivative of lipid A, particularly monophosphoryl lipid A or more particularly 3-Deacylated monophosphoryl lipid A (3D-MPL).

3D-MPL is sold under the name MPL by GlaxoSmithKline Biologicals N.A. and is referred to throughout the document as MPL or 3D-MPL. see, for example, U.S. Pat. Nos. 4,436,727; 4,877,611; 4,866,034 and 4,912,094. 3D-MPL primarily promotes CD4+ T cell responses with an IFN-g (Th1) phenotype. 3D-MPL can be produced according to the methods disclosed in GB 2 220 211 A. Chemically it is a mixture of 3-deacylated monophosphoryl lipid A with 3, 4, 5 or 6 acylated chains.

Other TLR-4 agonists which may be useful in the present invention are the aminoalkyl glucasminide phosphates (AGPs) which are synthetic TLR-4 agonists available from GlaxoSmithKline Biologicals S. A. Suitable examples are those disclosed in WO98/50399 or U.S. Pat. No. 6,303, 347 (processes for preparation of AGPs are also disclosed), suitably RC527 or RC529 or pharmaceutically acceptable salts of AGPs as disclosed in U.S. Pat. No. 6,764,840.

TLR7 and 8 are further members of the toll like receptor family. Small molecules are known that are agonists of either the TLR7 receptor or the TLR8 receptor or both. By TLR7/8 agonist is meant a molecule that can agonise (i.e. increase) the signalling of either the TLR7 receptor or the TLR8 receptor or both receptors. In one aspect therefore the TLR7/7 ligand is a molecule that is a TLR7 agonist but is not a TLR8 agonist. In another aspect, the TLR7/8 ligand is a TLR8 agonist but is not a TLR7 agonist. In a further aspect, the TLR7/8 ligand acts as an agonist at both the TLR7 and the TLR8 receptors. Suitable TLR7/8 ligands may be found for example in WO2010/018133, WO2010048520, WO2010/018134, WO 2008004948, WO 2007034882, and WO 2005092893.

It will be apparent to the skilled person as discussed further herein that some natural adjuvants may be present in the antigen preparation if such preparation is a live attenuated virus or a killed whole virus containing natural pathogen associated molecular patterns. In this context, the term “no other adjuvant” is not meant to exclude those natural adjuvants found in some antigenic preparations, but is intended to mean that no further adjuvants are specifically added to the immunogenic composition.

In one embodiment, the immunogenic composition of the present invention is used for cutaneous primary immunisation. In another embodiment, the immunogenic composition of the present invention is used for cutaneous booster immunisation in a subject who has undergone primary immunisation by a non-transcutaneous route, such as sublingually, intranasally or intramuscularly. In yet another embodiment, the immunogenic composition of the present invention may provide both cutaneous primary and cutaneous booster immunisation.

The term primary immunization is intended to mean the first course of vaccination that a subject receives against a particular pathogen. For example, in the UK vaccination schedule, infants are immunized against measles, mumps and Rubella at 13 months of age (a primary immunization). They are vaccinated again at 3 years and 4 months of age against the same pathogens (a booster immunization). Another example can be seen in the field of hepatitis B. People in need of vaccination (adults or infants) are given a primary schedule of three doses of vaccine at 0, 1 and 6 months (primary immunization). If necessary, (for example an accelerated primary schedule was followed, or antibody titres have decreased) another vaccination may be given at 1 year or 5 years following initial vaccination (booster immunization). A further example can be found in the so called “DTP” vaccines—diphtheria, tetanus, pertussis vaccines. In general, primary tetanus and diphtheria immunization is carried out during the first year of life in 2 doses. According to country, a booster dose is administered during the second year and/or between 4 and 10 years of age.

The term antigen is well understood in the art to mean an agent that produces an immune response. The antigen may be one or more proteins, polysaccharides, peptides, nucleic acids, protein-polysaccharide conjugates, molecules or haptens that are capable of raising an immune response in a human or animal. Alternatively the antigen may be a whole pathogen, for example an attenuated or inactivated pathogen. The whole inactivated pathogen may further be split, for example a split influenza virus. In one embodiment of the present invention, an antigen is derived from hepatitis A virus and/or hepatitis B virus (for example hepatitis B virus surface antigen). In another embodiment of the present invention, an antigen is derived from human papillomavirus. In another embodiment of the present invention, an antigen is nicotine, or is derived from nicotine. In another embodiment of the present invention, an antigen is derived from Dengue virus. In another embodiment of the present invention, an antigen is derived from Respiratory syncytial virus (RSV). In another embodiment the antigen is associated with Alzheimer's disease. In another embodiment the antigen is derived from the viruses causing measles, mumps, rubella or a combination thereof. In another embodiment the antigen is derived from Varicella Zoster Virus (VZV). In another embodiment the antigen is derived from a tumour associated antigen (for example MAGE and/or PRAME). In another embodiment the antigen is derived from a parasite that causes malaria in humans, in particular Plasmodium falciparum and/or Plasmodium vivax. In another embodiment the antigen is derived from cytomegalovirus (CMV).

If the immunogenic composition of the present invention is used as a booster immunisation, then the primary immunisation may have been either adjuvanted or not adjuvanted. It will be apparent that some vaccines naturally contain adjuvants, for example live attenuated or killed viral vaccines will retain some of the pathogen associated molecular patterns (PAMPS) that were to be found in the original pathogen. When the primary immunisation is “not adjuvanted”, this term is intended to mean that said primary immunisation does not contain any adjuvants in addition to those that may be present in the antigen preparation.

In one specific embodiment of the present invention, the primary immunisation is adjuvanted (i.e. additional adjuvants to those which may be naturally in the antigen preparation have been incorporated). An adjuvant is a term understood in the art to mean a component which assists in inducing an immune response to the antigen. Adjuvants useful for primary vaccination are, for example, metal salts, TLR modulators, oil in water emulsions, liposomal immunogenic composition, saponin adjuvants, or combinations of any of these.

In one embodiment, the adjuvant used in the primary immunisation comprises a TLR modulator, for example a TLR-4 modulator such as lipopolysaccharide or derivatives thereof for example monophosphoryl lipid A or 3-deacylated monophosphoryl lipid A (known as 3D-MPL, and available from GlaxoSmithKline Biologicals North America, see for example U.S. Pat. Nos. 4,436,727; 4,877,611; 4,866,034 and 4,912,094).

In another embodiment, the adjuvant used in the primary immunisation comprises a saponin adjuvant, for example Quil A and its derivatives. Quil A is a saponin preparation isolated from the South American tree Quillaja Saponaria Molina and was first described by Dalsgaard et al. in 1974 (“Saponin adjuvants”, Archiv. für die gesamte Virusforschung, Vol. 44, Springer Verlag, Berlin, p 243-254) to have adjuvant activity. Purified fragments of Quil A have been isolated by HPLC which retain adjuvant activity without the toxicity associated with Quil A (EP 0 362 278), for example QS7 and QS21 (also known as QA7 and QA21).

In one embodiment, the adjuvant used in the primary immunisation comprises both saponin adjuvant and a TLR-4 modulator, for example the adjuvant known as AS01 B (3D-MPL and QS21 in a liposomal immunogenic composition, 50 μg 3D-MPL and 50 μg QS21) or the adjuvant known as AS01E (3D-MPL and QS21 in a liposomal immunogenic composition, 25 μg 3D-MPL and 25 μg QS21).

In one embodiment, the adjuvant used in the primary immunisation comprises a metal salt such as aluminium hydroxide or aluminium phosphate and 3-deacylated monophosphoryl lipid A. In a specific example of this embodiment, the adjuvant used in the primary immunisation is the adjuvant known as AS04 (50 μg 3D-MPL adsorbed onto 500 μg aluminium salt).

In a further embodiment, the adjuvant used in the primary immunisation comprises an oil in water emulsion which itself comprises a metabolisable oil such as squalene and a surfactant such as Tween 80 and/or span 85. In a specific example of this embodiment, said oil-in-water emulsion is MF59. In one example of this embodiment, an oil-in-water emulsion may comprise a combination of metabolisable oils, such as squalene and alpha tocopherol. In a specific example of this embodiment, the oil-in-water emulsion adjuvant is AS03_{A}, AS03_{B}, AS03_{C} or AS03_{D} all of which are alpha-tocopherol based oil-in-water emulsions from GlaxoSmithKline Biologicals S.A.

### EXAMPLES

In order to assess the adjuvant potential of the native flagellin protein from Salmonella typhimurium (FliC protein), groups of C57BL/6 mice were injected intradermally on day 0 and on day 14 with either 2 μg of Hepatitis B surface antigen (HBsAg) alone or with HBsAg combined with increasing doses (0.001 μg, 0.01 μg or 0.1 μg) of native FliC protein from S. typhimurium. Control groups also received the same dose of HBsAg mixed with DQ adjuvant by the intradermal route, or adsorbed to 50 μg of alum via the intramuscular group. Mice were euthanized on day 28 and blood samples were collected by cardiac puncture. Blood samples were also collected prior each immunization. Blood samples were processed and serum samples frozen at −80° C. for antigen-specific antibody determination by ELISA. HBsAg was used as solid-phase antigen. Briefly, wells of microwell plates were coated for 4 hrs at room temp with an optimal concentration of HBsAg. Following washing and blocking of the microwells, serum samples were serially diluted into the plates and the plates were incubated overnight at 4° C. Following extensive washing steps, mouse IgG were detected using an HRP-conjugated secondary antibody (30 min at 37° C.) followed by incubation with TMB substrate solution. The reaction was stopped after 30 min with 1 M sulphuric acid. Plates were read at 450 nm. The antibody concentrations in the test samples were calculated from a standard curve run on each plate, using purified mouse antibodies. Specific serum IgG concentration was calculated from a standard by SoftMaxPro by using a four-parameter equation. Values were expressed as nanograms of specific antibody per milliliter of serum and means of antibody concentrations of the test groups were compared the control group having received unadjuvanted HBsAg intradermally (open circle on the graph) by one-way ANOVA followed by Dunnett's Multiple Comparison Test. Statistical analysis demonstrated an increase in immunogenicity showed by an increase in antigen-specific serum antibody concentration when HBsAg was co-injected with 0.1 μg of FliC in comparison to animals having received HBsAg alone. An increase in antibody concentration of approximately 7 folds was observed. Results from another separate experiment (data not shown) indicated that increasing the FliC dose to 1 μg does not further improve the immunogenicity of the HBsAg. Interestingly, there was no statistically significant difference between antibody titers from mice having received HBsAg with 0.1 μg of FliC and those from mice that received 1/10 of the human dose of Engerix vaccine (HBsAg 2 μg adsorbed to 50 μg of alum).

1. A patch comprising an immunogenic composition comprising one or more antigens and an adjuvant for use in cutaneous immunisation wherein said adjuvant is a TLR-5 agonist, wherein said patch comprises microprojections adapted to deliver immunogenic composition of the invention to the epidermis, and wherein at least part of the microprojections is biodegradable.

2. The patch according to claim 1 wherein said immunogenic composition further comprises one or more additional adjuvants selected from the group consisting of: TLR-4 agonists, TLR7/8 agonists, or an immunologically active saponin.

3. The patch according to claim 1 which is administered in the form of a patch.

4. The patch according to claim 1 which is administered using a short needle device.

5. The patch according to claim 1 wherein said TLR-5 agonist is a flagellin or a fragment thereof having TLR-5 activity.

6. The patch according to claim 5 wherein said TLR-5 agonist is one in which the hypervariable domain has been deleted.

7. The patch according to claim 5 in which said TLR-5 agonist is selected from the group consisting of: FliC_{Δ174-400}; FliC_{Δ161-405} and FliC_{Δ138-405}.

8. The patch according to claim 1 wherein one of the antigens is Hepatitis B surface antigen (HBsAg).
